(19) United States
(12) Patent Application Publication
(10) Pub. No.: US 2021/0065255 A1
(43) Pub. Date: Mar. 4, 2021

(54) MATERIAL PROPERTY RATING SYSTEM AND MATERIAL PROPERTY RATING METHOD

(71) Applicant: INDUSTRIAL TECHNOLOGY RESEARCH INSTITUTE, Hsinchu (TW)

(72) Inventors: Che-Ming Chang, Hsinchu (TW); Yen-Ting Li, Hsinchu (TW); Kuo-Chan Chiou, Hsinchu (TW); Chun-Wei Su, Hsinchu (TW); Shi-Yun Chern, Hsinchu (TW)

(21) Appl. No.: 17/009,820

(22) Filed: Sep. 2, 2020

Related U.S. Application Data
(60) Provisional application No. 62/895,022, filed on Sep. 3, 2019.

(30) Foreign Application Priority Data
Aug. 11, 2020 (TW) 109127221

(51) Int. Cl. G06Q 30/02; G06Q 40/02; G06N 20/00; G06F 16/22

(52) U.S. Cl. CPC G06Q 30/0278; G06F 16/22; G06N 20/00; G06Q 40/025

(57) ABSTRACT

A material property rating method and a material property rating system are provided, which analyze the reliability of a target information of a target object provided by a target source through an analysis module, and then calculates the credibility of the target source based on the reliability of the target information through a credit rating module. Therefore, when the material property rating system is applied to a material information platform, the material property rating system can effectively avoid false information, and can save time and effort for the verification process, and can be trusted by consumers browsing the material information platform.

### BACKGROUND

### 1. Technical Field

The present disclosure generally relates to a rating system and a rating method. More specifically, the present disclosure relates to a material property rating system and material property rating method for the credibility of vendors or reliability of the material property.

### 2. Description of Related Art

With the development of network era, many information are integrated and published on online platforms for user reference.

Big data and digitized technologies have been undergoing rapid development, material industry has thus incorporated these relevant technologies to set up material information platforms to facilitate product design and material development.

In a conventional material information platform, suppliers provide the materials and products they sell, and assert the properties of these materials and products.

However, to ensure the information published on the material information platform is reliable or trustable, the developer of the material information platform is required to verify the properties of the items provided by the suppliers one by one to avoid false information. However, such a method not only takes effort and time, but does not easily gain the trust of consumers who are actually browsing the material information platform.

### SUMMARY

In order to address the aforementioned shortcomings, a material property rating system is disclosed, which may include an analysis module for receiving a target information of a target object provided by a target source to analyze a reliability of the target information; and a credit rating module communicatively connected with the analysis module to calculate a credibility of the target source based on the reliability of the target information.

A method of rating material property is further disclosed, which may include: analyzing a reliability of a target information of a target object provided by a target source; and determining whether the reliability of the target information satisfies a preset condition so as to decide if a credibility of the target source is to be calculated, wherein if the reliability of the target information does not satisfy the preset condition, then the credibility of the target source is calculated based on the reliability of the target information.

As can be understood from the above, in the material property rating system and the material property rating method of the present disclosure, the reliability of the target information is analyzed, and the credibility of the target source can be calculated based on the reliability of the target information. This automatically verifies the reliability of the target information to provide credibility of the target source. Thus, compared to the prior art, the material property rating system of the present disclosure, when applied to a material information platform, eliminates false information and can be trusted by consumers browsing the material information platform, while having a verification process that saves time and effort.

### BRIEF DESCRIPTION OF THE DRAWINGS

FIG. 1 is a schematic view depicting the architecture of a material property rating system in accordance with the present disclosure.

FIG. 2 is a schematic view depicting configuration of a material property rating system in accordance with the present disclosure.

FIG. 3A is a schematic view depicting the operations of an analysis module of a material property rating system in accordance with the present disclosure.

FIG. 3B is a schematic view depicting the operations of a determining module of a material property rating system in accordance with the present disclosure.

FIG. 4 is a flowchart illustrating a material property rating method in accordance with the present disclosure.

### DETAILED DESCRIPTION

Embodiments of the present disclosure are explained with specific implementations below. Other advantages and technical effects of the present disclosure can be readily understood by one of ordinary skill in the art upon reading the disclosure provided herein.

It should be noted that the structures, ratios, sizes shown in the drawings appended to this specification are to be construed in conjunction with the disclosure of this specification in order to facilitate understanding of those skilled in the art. They are not meant, in any ways, to limit the implementations of the present disclosure, and therefore have no substantial technical meaning. Without affecting the effects created and objectives achieved by the present disclosure, any modifications, changes or adjustments to the structures, ratio relationships or sizes, are to be construed as fall within the range covered by the technical contents disclosed herein. Meanwhile, terms, such as “above,” “below,” “first,” “second,” “third,” “one,” “a,” “an,” and the like, are for illustrative purposes, and are not meant to limit the range implementable by the present disclosure. Any changes or adjustments made to their relative relationships, without modifying the substantial technical contents, are also to be construed as within the range implementable by the present disclosure.

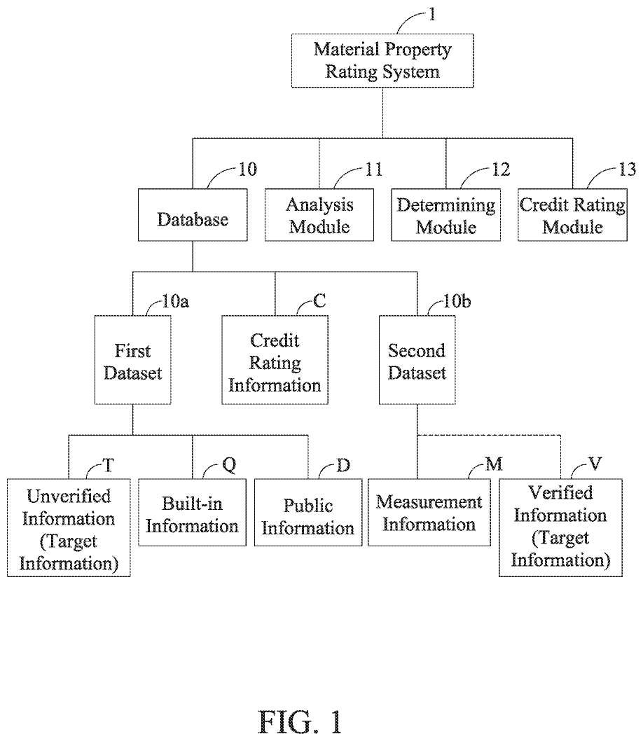

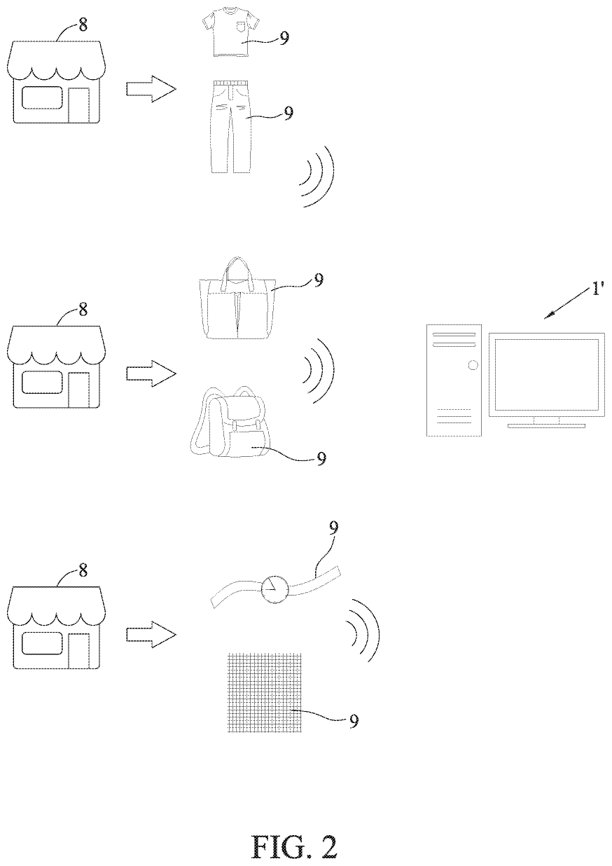

FIG. 1 is a schematic view depicting the architecture of a material property rating system 1 in accordance with the present disclosure. As shown in FIG. 1, the material property rating system 1 is configured within an electronic apparatus 1′ (shown in FIG. 2), such as a computer, and includes a database 10, an analysis module 11, a determining module 12 and a credit rating module 13.

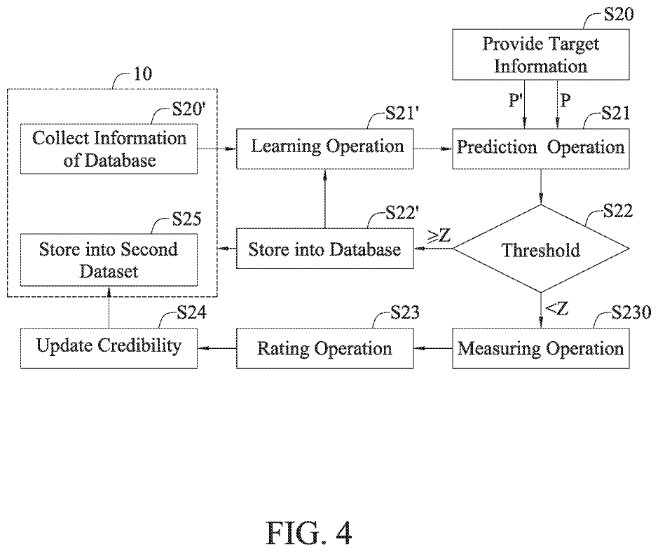

A first dataset 10a and a second dataset 10b are stored in the database 10. The first dataset 10a are data that have not yet been verified (e.g., not undergone a measuring operation in step S230 of FIG. 4 described later), and the second dataset 10b are data that have been verified (e.g., completed the measuring operation in step S230 of FIG. 4).

In an embodiment, the first dataset 10a includes at least one unverified information T, which specifies the properties of a target object 9 (as shown in FIG. 2), and the target object 9 can be, for example, a flexible object, such as a product or its raw materials (such as leather, cloth, fabric, or other materials), so the unverified information T contains the properties of the flexible object, with items such as stretchability, bendability, heat resistance, impact resistance, sweat corrosion resistance, biocompatibility, material adhesion, tensile recoverability, skin affinity, UV resistance, washing resistance, water resistance, high temperature cycling impact test, high temperature and high humidity test or other material characteristics. It should be understood that there are many types of target object 9 and are not limited to flexible objects. Therefore, the items for the properties of the unverified information T may also be different, and the unverified information T may also include a measuring method for obtaining each property to facilitate the learning of the analysis module 11.

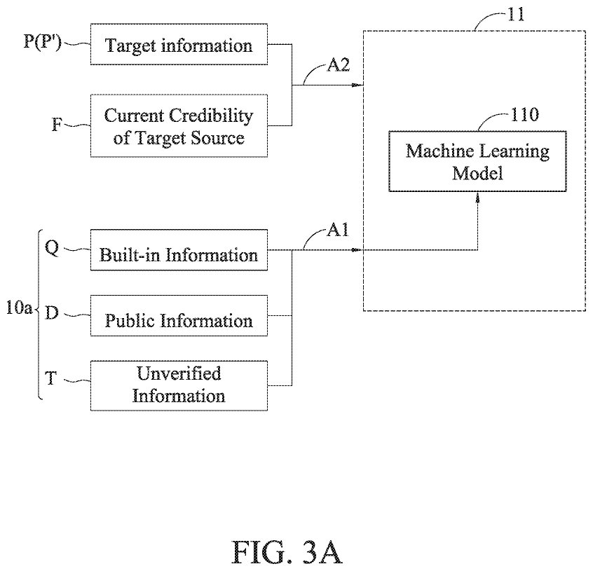

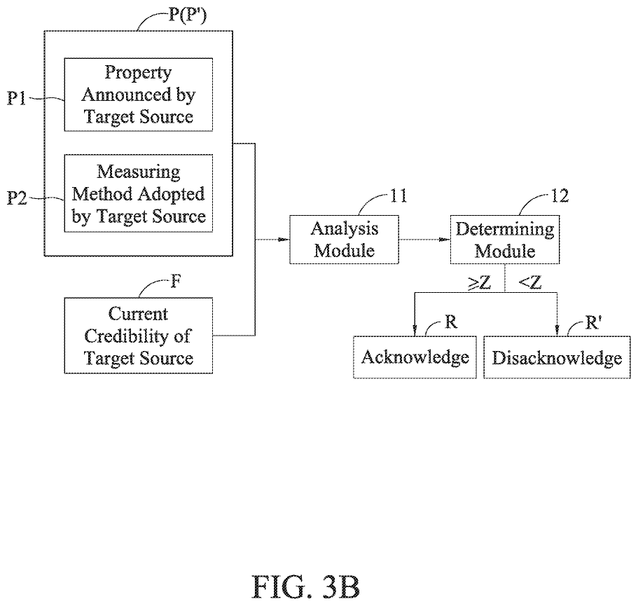

Moreover, the target object 9 comes from, for example, a target source 8 (as shown in FIG. 2), such as the supplier end (e.g., a supplier or personnel) or the manufacturing end (e.g., a manufacturer or personnel). Thus, the unverified information T is provided by the target source 8 (that is, announced by the supplier end or the manufacturing end themselves, such as a property P1 announced by target source as shown in FIG. 3B). More specifically, a single target source 8 may provide at least one target object 9 or input at least one unverified information T into the database 10, so that the unverified information T of at least one target object 9 and the target source 8 to which it belongs are stored in the database 10.

Moreover, the first dataset 10a can also include built-in information Q and/or public information D, which can both correspond to the properties of the target object 9. The built-in information Q is the properties of the metric standards, that is, the properties obtained by adopting the universal standards, and the public information D is information from sources such as scientific literature, scientific journals, patents, books, company announcements, or others. Standards for various properties are, for example, ASTM D638 for measuring stretchability, ASTM D790 for measuring bendability, IPC-TM-650 for measuring heat resistance, ASTM D4966 for measuring impact resistance, ISO 12870 for measuring sweat corrosion resistance, ISO 10933 for measuring biocompatibility, ASTM D3359 for measuring material adhesion, ISO 4892-1 for measuring UV resistance, JESD22-A104 for high temperature cycling impact test, JESD22-A101 for high temperature and high humidity test, etc. It should be understood that there are many types of norms and standards related to various properties, which are well-known to those skilled in the related fields, so will not be illustrated further.

In addition, a credit rating information C is further stored in the database 10, which contains data of the credibility of each of the target sources 8, and the data of the credibility of each of the target sources 8 can be updated constantly.

The analysis module 11 receives at least one target information P of the target object 9 provided by the target source 8 and analyzes the reliability of the target information P. The reliability of the target information P is analyzed through an artificial intelligence model.

In an embodiment, the analysis module 11 carries out machine learning training by using the first dataset 10a in order to analyze the reliability of the target information P. For example, the analysis module 11 includes a machine learning model 110 (as indicated by a learning phase A1 in FIG. 3A), such as a Support Vector Machine (SVM) model, a neural network (NN) training model, a Random Forest model, a k-Nearest Neighbors (KNN) algorithm or other artificial intelligence (AI) models. The model uses the first dataset 10a of the database 10 as its input to output predictions (e.g., reliable or not reliable) of various reliabilities of the first dataset 10a, and undergoes continuous training through a classification algorithm. As such, after a target information P as well as a current credibility F of the target source 8 to which the target information P belongs are inputted into the analysis module 11 (as indicated by a prediction phase A2 in FIG. 3A), the analysis module 11 can then quickly predict the reliability of the target information P.

Moreover, the target information P are properties of the target object 9, which is for example, a flexible object, such as a product or its raw materials (e.g., leather, cloth, fabric, or other materials), so the target information P contains the properties of the flexible object, with items such as stretchability, bendability, heat resistance, impact resistance, sweat corrosion resistance, biocompatibility, material adhesion, tensile recoverability, skin affinity, UV resistance, washing resistance, water resistance, high temperature cycling impact test, high temperature and high humidity test or other material characteristics. It should be understood that there are many types of target object 9 and are not limited to the above. Therefore, the items for the properties of the target information P may also be different, and the target information P may also include a measuring method (e.g., measuring method P2 adopted by target source in FIG. 3B) for obtaining each property to facilitate verification of the property.

Furthermore, the target information P is provided by the target source 8, in other words, announced by the supplier or manufacturing end, and a single target source 8 may provide at least one target object 9 or input at least one target information P to the analysis module 11.

The determining module 12 is communicatively connected with the analysis module 11 to determine if the reliability of the target information P satisfies a preset condition, which allows the credit rating module 13 to determine whether to perform algorithms.

In an embodiment, the preset condition is set by the material property rating system 1. For example, the preset condition is defined as a threshold Z, which serves as an acknowledgement threshold. More specifically, as shown in FIG. 3B, when the reliability of the target information P is analyzed by the analysis module 11, and is determined to be less than the threshold Z by the determining module 12, then a determining report R′ of the determining module 12 does not acknowledge the target information P. As a result, the target information P will not be stored directly into the database 10, and further verification is needed to be able to decide whether the target information P is to be stored in the database 10. On the other hand, when the reliability of the target information P is determined by the determining module 12 to be greater than or equal to the threshold Z, then the determining report R acknowledges the target information P, and the target information P is directly stored into the first dataset 10a of the database 10 to be used as unverified information T. Therefore, the unverified information T of the first dataset 10a may be directly inputted into the database 10 by the target source 8 or come from the target information P provided by the determining module 12.

Moreover, the material property rating system 1 can adjust the threshold Z (e.g., based on the property item of the target information or the current credibility F of the target source 8), so that the reliability of the same target information P may return two different results (disacknowledgement in report R′ and acknowledgement in report R). For example, the reliability of the target information P is 70%, and the threshold Z is 80%, then the determining report R′ returns a disacknowledgement. If, on the other hand, the threshold Z is 40%, then the determining report R returns an acknowledgement. Therefore, whether or not the target information P can be directly stored in the database 10 will depend on the magnitude of the threshold Z set by the material property rating system 1.

The credit rating module 13 is communicatively connected with the analysis module 11 or communicatively connected with the determining module 12 if needed to calculate the credibility F of the target source 8 based on the reliability of the target information P.

In an embodiment, the credit rating module 13 calculates the credibility of the target source 8 using a measurement information M in conjunction with the target information P. For example, the measurement information M is a reference data obtained by measuring the target object 9, and is classified as the second dataset 10b. The measurement information M corresponds to the properties of the flexibility object in the target information P, such as stretchability (using ASTM D638 standard), bendability (using ASTM D790 standard), heat resistance (using IPC-TM-650 standard), impact resistance (using ASTM D4966 standard), sweat corrosion resistance (using ISO 12870 standard), biocompatibility (using ISO 10933 standard), material adhesion (using ASTM D3359 standard), stretch recoverability (using related standards), skin affinity (using related standards), UV resistance (using ISO 4892-1 standard), washing resistance (using relevant standards), water resistance (using relevant standards), high temperature cycling impact test (using JESD22-A104 standard), high temperature and high humidity test (using JESD22-A101 standard), or other items obtained by appropriate specifications or standards. Thus, the credibility of the target source 8 can be determined based on how great the difference is between the measurement information M and the target information P. More specifically, the smaller the difference between the measurement information M and the target information P, the more credible the target source 8 is.

In addition, the credibility of the target source 8 is presented as a relative numerical value, that is, relative to a baseline score. For example, a rating function below can be used by the credit rating module 13 to calculate the credibility of the target source 8:

$${S}_{posterior} = {S}_{prior} - {{Σ}_{i}^{n} ( {NorR}_{verified , i} - {NorR}_{unverified , i} )}^{2} - c or$$

$${S}_{posterior} = {S}_{prior} - {Σ}_{i}^{n} ( \frac{{( {y}_{truth , i} - {y}_{announced , i} )}^{2}}{{\overline{y}}_{i}} - c )$$

wherein Sₚₒₛₜₑᵣᵢₒᵣ is the new credibility score of the target source 8 obtained after calculation (or after update); Sₚᵣᵢₒᵣ is the baseline score or the previous credibility score of the target source 8; NorR_{verified,i} is the verified property of an in, item in the database 10 after normalization; NorR_{unverified,1} is the unverified property of the iₜₕ item in the database 10 after normalization; n is the number of items for the property of the target information P of the target object 9; y̅ᵢ is an average value corresponds to the property of the iₜₕ item in the database 10; y_{truth,i} is the value of the property of the iₜₕ item of the measurement information M; y_{announced,i} is the value of the property of the in, item of the target information P; and c is a constant that can be arbitrary adjusted (for controlling the sensitivity of the rating function). More specifically, the baseline score (Sₚᵣᵢₒᵣ) can be 0, 0.5 or 1.0, and a plurality of target sources 8 are various vendors as shown in the table below:

| Target Source | Taipei Vendor | Taichung Vendor | Tainan Vendor |
|---|---|---|---|
| Score (baseline | 1.3 | 1.5 | 0.4 |
| score = 1.0) | | | |

wherein the higher the credibility score of a particular target source 8 is to the baseline score, the better the credibility, and the lower the credibility score is to the baseline score, the worse the credibility of the target source 8. This can be used by the material property rating system 1 to determine whether the target object 9 and its target information P provided by the target source 8 are to be stored in the database 10, or allow a consumer when purchasing the target object 9 using the database 10 to know the credibility of the target source 8 to which the target object 9 belongs.

Alternatively, the credibility of the target source 8 can be presented as grades, for example, as shown in the table below:

| Target Source | Taipei Vendor | Taichung Vendor | Tainan Vendor |
|---|---|---|---|
| Grade | A | A | C |

wherein if the credibility grade of the target source 8 is A, then the target source 8 has better credibility. If it is not A (e.g., B or C), then the target source 8 has a poorer credibility. This can be used by the material property rating system 1 to determine whether the target object 9 and its target information P provided by the target source 8 are to be stored in the database 10, or allow a consumer when purchasing the target object 9 using the database 10 to know the credibility of the target source 8 to which the target object 9 belongs.

It can be appreciated that there are numerous ways of presenting credibility depending on the needs, and the present disclosure is not limited to the above.

FIG. 4 is a flowchart illustrating a material property rating method in accordance with the present disclosure. In this embodiment, the method is performed using the material property rating system 1 above.

In step S20, a target source 8 provides a target information P of a target object 9 to the analysis module 11, wherein the target source 8 can be a new vendor or one already recorded in the database 10, and the target information P of the target object 9 is the property data announced by the target source 8 (e.g., property P1 announced by target source shown in FIG. 3B), that is, new unverified data.

In an embodiment, the target information P includes properties of several items and their measuring methods as indicated in the table below:

| Item | Value | Measuring Method |
|---|---|---|
| First Property | 330 | ASTM-E1356 |
| Second Property | 25% | ASTM D638 |

wherein the first property is, for example, the glass transition temperature (Tg), and the second property is, for example, the maximum elongation.

Then, if needed, the property data (such as a first dataset 10a) corresponding to the target information P are already stored in the database 10 and can be provided to the analysis module 11 to perform a learning process, such as that shown in steps S20′-S21′. It can be appreciated that if the machine learning model 110 of the analysis module 11 has already completed the related training, then steps S20′-S21′ need not be performed.

In step S21, prediction operations are performed, in which reliability of the target information P is analyzed and predicted by the analysis module 11.

In an embodiment, the analysis module 11 outputs a prediction report on reliability after analysis as shown below:

| Item | Prediction Result | Reason for Discrepancy |
|---|---|---|
| First Property | 8 | Omitted |
| Second Property | 3 | Omitted |

wherein the prediction report shows the discrepancy (i.e., the prediction result) between the property of each item of the target information P and the existing data of the database 10 and the reason for such discrepancy.

In step S22, whether or not the reliability or trustability of the target information P satisfies a preset condition (e.g., a threshold Z) is determined by the determining module 12. Then, whether or not calculation on the credibility of the target source 8 is determined.

In an embodiment, in step S22′, if the threshold Z is 7, and when the reliability of the first property of the target information P is greater than or equal to the threshold Z, then the first property of the target information P does not need to be verified and can be directly stored in the database 10, as shown in the table below, to be classified as the first dataset 10a (shown in FIG. 1) to be used by the machine learning model 110.

| Prediction of Reliability | Prediction Result | Verification |
|---|---|---|
| First Property | 8 (≥7) | Not required |
| Second Property | 3 (<7) | Required |

Moreover, the material property rating system 1 can adjust the threshold Z based on the reason of the discrepancy. For example, the reason of the discrepancy may be due to the different measuring methods used, so that the property of the target information P is different from the existing data of the database 10. Thus, the material property rating system 1 can lower the acknowledgement threshold (i.e., the threshold Z). Alternatively, in view of a high-end precision standard of the database 10, the material property rating system 1 can raise the acknowledgement threshold (i.e., the threshold Z), so that the target information P stored in the database 10 is very precise.

In step S23, the credibility of the target source 8 is calculated by the credit rating module 13 based on the reliability of the target information P.

In an embodiment, the credibility of the target source 8 can be calculated using a measurement information M in conjunction with the target information P. For example, as shown in the table above, when the reliability of the second property of the target information P is less than the threshold Z, the material property rating system 1 needs to verify the second property, as shown by a measuring step shown in step S230, so as to verify the target information P to obtain the measurement information M for calculation of the credibility of the target source 8. For example, the second property of the target information P is 25%, the maximum elongation corresponding to the measurement information M is 15%, and an average value corresponding to the maximum elongation item in the database 10 is 17%. Using the rating function described above and with the previous credibility of the target source 8 being 0.85 and the constant c set to 0.03, the credibility score of the target source 8 after calculation (or after update) is 0.82. The calculation is shown below:

$${S}_{posterior} = 0.85 - ( \frac{{( 0.15 - 0.25 )}^{2}}{0.17} - 0.03 ) = 0.85 - 0.03 = 0.82$$

As can be seen, since the discrepancy between the second property specified by the target information P (25%) and the measurement information M is too great, the credibility of the target source 8 is therefore affected and downgraded, that is, the target source 8 now has poorer credibility.

In steps S24-S25, the credibility of the target source 8 calculated by the credit rating module 13 is updated and stored in the database 10 along with the measurement information M, wherein the measurement information M is classified under the second dataset 10b, so when a consumer is purchasing the target object 9 using the database 10, the consumer will know the credibility of the target source 8 to which the target object 9 belongs.

In an embodiment, since the credibility of the target source 8 is downgraded after calculation, if needed, the material property rating system 1 can decide whether to store the second property of the target information P into the database 10 after reviewing the credibility of the target source 8.

In another embodiment, the target source 8 provides a target information P′ of another target object 9, which upgrades its credibility. Details are set forth below.

In step S20, the target information P′ includes properties of three items and their measuring methods as indicated in the table below:

| Item | Value | Measuring Method |
|---|---|---|
| First Property | 330 | ASTM-E1356 |
| Second Property | 25% | ASTM D638 |
| Third Property | 35 cm | ASTM D790 |

wherein the first property is Tg, the second property is the maximum elongation, and the third property is the bend radius.

In step S21-S230, if the threshold Z is 7, then the first and second properties need to be verified by the material property rating system 1. This is indicated in the table below:

| Prediction of Reliability | Prediction Result | Verification |
|---|---|---|
| First Property | 4 (<7) | Required |
| Second Property | 3 (<7) | Required |
| Third Property | 7 (≥7) | Not required |

wherein the Tg value corresponding to the measurement information M is 328, and the average value corresponding to the Tg item in the database 10 is 345, and the maximum elongation corresponding to the measurement information M is 18%.

In step S23, using the rating function described above, with the current credibility of the target source 8 being 0.35 and the constant c set to 0.03, the credibility score of the target source 8 after calculation (or after update) is 0.40. The calculation is shown below:

$$\begin{matrix}{S}_{posterior} = 0.35 - ( \frac{{( 328 - 330 )}^{2}}{345} - 0.03 ) - ( \frac{{( 0.18 - 0.20 )}^{2}}{0.17} - 0.03 ) \\ = 0.35 - ( - 0.02 ) - ( - 0.03 ) = 0.40\end{matrix}$$

Accordingly, the credibility of the target source 8 after calculation has improved, so the material property rating system 1 can, after considering the credibility of the target source 8 after calculation (or after update), decide according to the needs whether to store the first and second properties of the target information P′ into the database 10 as the second dataset 10b (i.e., the verified information shown in FIG. 1).

In conclusion, the material property rating system 1 and the material property rating method of the present disclosure automatically predicts the reliability of the target information P, P′ through the analysis module 11 to quickly ascertain the discrepancy (i.e., prediction result) between the property of each item of the target information P, P′ and the existing data of the database 10 and the reason for such discrepancy, so as to determine if the property of each item of the target information P, P′ needs to be verified one by one, for example, using the acknowledgement threshold (i.e., the threshold Z). Therefore, the reliability of the material data of the target information P, P′ can be quickly determined (if the acknowledgement threshold is satisfied, then no measurement verification is needed, and the material data is directly added to the first dataset 10a of the database 10 to reduce the need to repeatedly verifying each material data, as shown in steps S22-S22′; else if the acknowledgement threshold is not satisfied, then verification is required, as shown by the measuring step of step S230). Therefore, this allows the proprietor of the material property rating system 1 to avoid false information while reducing the number of items that requires verification, thereby achieving quicker and more efficient verification.

In addition, the credibility of the target source 8 can be calculated by the credit rating module 13 based on the reliability of the target information. This facilitates the building of the database 10, and increases the reliability of the target information P, P′, so the database 10 allows consumers to obtain the credibility of the target source 8 to which the target object 9 belongs, gaining the trust of the consumers and also more extensive use of the database 10.

While embodiments of the present disclosure have been disclosed in detail herein, it should be appreciated that the present disclosure is not limited thereto or thereby inasmuch as variations on the disclosure herein will be readily appreciated by those of ordinary skill in the art. The scope of the present disclosure shall be appreciated from the claims that follow.

What is claimed is:

1. A material property rating system, comprising:
an analysis module for receiving a target information of a target object provided by a target source to analyze a reliability of the target information; and
a credit rating module communicatively connected with the analysis module to calculate a credibility of the target source based on the reliability of the target information.

2. The material property rating system of claim 1, wherein the analysis module performs machine learning training using a database so as to analyze the reliability of the target information, and the database includes an unverified data for use by the analysis module in the machine learning training.

3. The material property rating system of claim 1, wherein the credit rating module calculates the credibility of the target source using a measurement information in conjunction with the target information, and the measurement information is calculation data obtained from measuring the target object.

4. The material property rating system of claim 1, further comprising a determining module communicatively connected with the analysis module and the credit rating module for determining whether the reliability of the target information satisfies a preset condition, so as to decide if the credit rating module will perform calculation of the credibility of the target source or not.

5. The material property rating system of claim 4, further comprising a database, wherein the database is stored with a first dataset and a second dataset, and wherein the first dataset includes an unverified information, and the second dataset includes a verified information.

6. The material property rating system of claim 5, wherein the unverified information of the first dataset is directly stored by the target source into the database, or from the target information provided by the determining module.

7. The material property rating system of claim 5, wherein after the credibility of the target source is calculated, the target information provided by the target source is stored in the database and classified as the second dataset.

8. The material property rating system of claim 1, wherein the credibility of the target source is a grade or a relative numerical value.

9. A method of rating material property, comprising:
analyzing a reliability of a target information of a target object provided by a target source; and
determining whether the reliability of the target information satisfies a preset condition so as to decide if a credibility of the target source is to be calculated,
wherein if the reliability of the target information does not satisfy the preset condition, then the credibility of the target source is calculated based on the reliability of the target information.

10. The method of claim 9, wherein the reliability of the target information is analyzed using an artificial intelligence model, and the artificial intelligence model uses a database to perform machine learning training, and the database includes an unverified information for use by the artificial intelligence model in performing the machine learning training.

11. The method of claim 10, wherein the database is stored with a first dataset and a second dataset, and wherein the first dataset includes the unverified information, and the second dataset includes a verified information.

12. The method of claim 11, wherein the unverified information of the first dataset is directly stored by the target source into the database, or from the target information provided by a determining module.

13. The method of claim 11, wherein after the credibility of the target source is calculated, the target information provided by the target source is stored in the database and classified as the second dataset.

14. The method of claim 9, wherein the credibility of the target source is calculated using a measurement information in conjunction with the target information, and the measurement information is calculation data obtained from measuring the target object.

15. The method of claim 9, wherein the credibility of the target source is a grade or a relative numerical value.
